(19) United States
(12) Patent Application Publication
(10) Pub. No.: US 2022/0039644 A1
(43) Pub. Date: Feb. 10, 2022

(54) DEVICES AND METHODS FOR TREATING A STRICTURE ALONG THE BILIARY AND/OR PANCREATIC TRACT

(71) Applicants: BOSTON SCIENTIFIC SCIMED, INC., Maple Grove, MN (US); NORTHWESTERN UNIVERSITY, Evanston, IL (US)

(72) Inventors: PETER L. DAYTON, Brookline, MA (US); RAYMOND D. GESSLER, III, St. Paul, MN (US); SRINADH KOMANDURI, Downers Grove, IL (US)

(73) Assignees: BOSTON SCIENTIFIC SCIMED, INC., Maple Grove, MN (US); NORTHWESTERN UNIVERSITY, Evanston, IL (US)

(21) Appl. No.: 17/392,847

(22) Filed: Aug. 3, 2021

Related U.S. Application Data
(60) Provisional application No. 63/061,469, filed on Aug. 5, 2020.

(51) Int. Cl. A61B 1/273; A61B 1/00; A61B 17/00; A61B 17/34

(52) U.S. Cl. CPC A61B 1/2736; A61B 1/00135; A61B 17/00234; A61B 2017/00336; A61B 2017/00296; A61B 2017/00309; A61B 2017/00323; A61B 17/3478

(57) ABSTRACT

Medical devices for use along the biliary and/or pancreatic tract. An example system for treating a stricture, for example along the biliary and/or pancreatic tract, may include a guidewire for antegrade stricture crossing along the biliary and/or pancreatic tract. The guidewire may have a distal end region and a proximal end region. A hub may be coupled to the proximal end region. A tubular sheath may be slidably disposed along the guidewire. The tubular sheath may have a plurality of slots formed therein. The hub may be configured to secure the axial position of the tubular sheath relative to the guidewire.

### CROSS REFERENCE TO RELATED APPLICATIONS

This application claims the benefit of and priority to U.S. Provisional Patent Application Ser. No. 63/061,469 filed on Aug. 5, 2020, the disclosure of which is incorporated herein by reference.

### TECHNICAL FIELD

The present disclosure pertains to medical devices, and methods for manufacturing medical devices. More particularly, the present disclosure pertains to medical devices for treating strictures along the biliary and/or pancreatic tract.

### BACKGROUND

A wide variety of medical devices have been developed for medical use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. A system for treating a stricture is disclosed. The system comprises: a guidewire for antegrade stricture crossing along the biliary and/or pancreatic tract, the guidewire having a distal end region and a proximal end region; a hub coupled to the proximal end region; a tubular sheath slidably disposed along the guidewire, the tubular sheath having a plurality of slots formed therein; and wherein the hub is configured to secure the axial position of the tubular sheath relative to the guidewire.

Alternatively or additionally to any of the embodiments above, further comprising an introducer sheath, wherein the guidewire is configured to be advanced through the introducer sheath.

Alternatively or additionally to any of the embodiments above, further comprising an endoscope, wherein the guidewire is configured to be advanced through the endoscope.

Alternatively or additionally to any of the embodiments above, the tubular sheath has a proximal portion having a first slot density and a distal portion having a second slot density greater than the first slot density.

Alternatively or additionally to any of the embodiments above, the tubular sheath has a proximal region free of slots.

Alternatively or additionally to any of the embodiments above, the plurality of slots define a plurality of beams including a first beam and a plurality of rings including a first ring in the tubular sheath, wherein the first beam has a first beam height, wherein the first ring has a first ring length, and where the first beam height is substantially equal to the first ring length.

Alternatively or additionally to any of the embodiments above, the hub includes a locking member for securing the axial position of the tubular sheath relative to the guidewire.

Alternatively or additionally to any of the embodiments above, the hub is secured to the tubular sheath.

Alternatively or additionally to any of the embodiments above, the tubular sheath defines a contrast lumen for infusing contrast media.

A method for treating a stricture along the biliary and/or pancreatic tract is disclosed. The method comprises: advancing an introducer sheath through a working channel of an endoscope to a position along the biliary and/or pancreatic tract to a position adjacent to a stricture; advancing a multi-part guidewire system through the introducer sheath, the multi-part guidewire system comprising: a guidewire having a distal end region and a proximal end region, a hub coupled to the proximal end region, a tubular sheath slidably disposed along the guidewire, the tubular sheath having a plurality of slots formed therein, and wherein the hub is configured to secure the axial position of the tubular sheath relative to the guidewire; shifting the axial position of the tubular sheath relative to the guidewire; and advancing the multi-part guidewire system past the stricture.

Alternatively or additionally to any of the embodiments above, advancing an introducer sheath through a working channel of an endoscope to a position along the biliary and/or pancreatic tract to a position adjacent to a stricture includes piercing through tissue.

Alternatively or additionally to any of the embodiments above, advancing an introducer sheath through a working channel of an endoscope to a position along the biliary and/or pancreatic tract to a position adjacent to a stricture includes an antegrade approach toward the stricture.

Alternatively or additionally to any of the embodiments above, further comprising removing the tubular sheath from the guidewire.

Alternatively or additionally to any of the embodiments above, further comprising advancing a treatment device over the guidewire.

Alternatively or additionally to any of the embodiments above, a portion of the guidewire extends distally beyond a distal end of the tubular sheath, and wherein shifting the axial position of the tubular sheath relative to the guidewire includes lengthening the portion of the guidewire that extends distally beyond the distal end of the tubular sheath.

Alternatively or additionally to any of the embodiments above, a portion of the guidewire extends distally beyond a distal end of the tubular sheath, and wherein shifting the axial position of the tubular sheath relative to the guidewire includes shortening the portion of the guidewire that extends distally beyond the distal end of the tubular sheath.

A system for treating a stricture along the biliary and/or pancreatic tract is disclosed. The system comprises: a support sheath having a distal end region, a proximal end region, and having a plurality of slots formed therein; a hub secured to the proximal end region; a guidewire slidably disposed within the support sheath; and wherein the hub is configured to secure the axial position of the guidewire relative to the support sheath.

Alternatively or additionally to any of the embodiments above, the support sheath has a proximal portion having a first slot density and a distal portion having a second slot density greater than the first slot density.

Alternatively or additionally to any of the embodiments above, the support sheath has a proximal region free of slots.

Alternatively or additionally to any of the embodiments above, the plurality of slots define a plurality of beams including a first beam and a plurality of rings including a first ring in the tubular sheath, wherein the first beam has a first beam length, wherein the first ring has a first ring length, and where the first beam length is substantially equal to the first ring length.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a side view of a medical device according to the present disclosure.

FIG. 2 is a side view of a medical device according to the present disclosure.

FIG. 3 is a side view of a portion of a medical device according to the present disclosure.

FIG. 4 is a side view of a medical device system according to the present disclosure.

FIGS. 5-8 illustrate a method for using a medical device system according to the present disclosure.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

In endoscopy, a frequent medical condition arises when a patient presents with abdominal pain with or without associated jaundice. The etiology is usually some type of obstruction in the biliary tree which prevents bile from flowing naturally from the proximal tree into the duodenum. The blockage may be the result of biliary stones caught in the lumen of the ducts or a tumor which is either in the wall of the duct or impinging upon the wall from adjacent tissue. When such a stricture occurs the duct proximal to the stricture dilates and the duct distal to the stricture receives a reduced flow of bile. In order to relieve the patient's symptoms, gastroenterologists seek to find a method for resuming the flow of bile from the proximal dilated duct into the duodenum. Some interventions contemplated for reliving symptoms may include placing a stent across the stricture to drain the proximal duct, removing a stone, and/or the like.

The most common method of placing a stent across the stricture is to perform an endoscopic retrograde cholangio-pancreatography (ERCP) where a side-viewing endoscope is placed in the duodenum at the location of the biliary papilla and a guidewire is placed through the papilla and up the biliary duct, across the stricture, in a retrograde fashion. Such procedures may be challenging. For example, depending on the location, geometry, and mechanics of the stricture, deep cannulation of the proximal duct may be difficult if not be possible. Furthermore, when the physician attempts to access the biliary duct, they may inadvertently cannulate the pancreatic duct. Inadvertent cannulation of the pancreatic duct could lead to complications such as pancreatitis. Disclosed herein are devices and methods that address these and other issues, for example by utilizing antegrade (e.g., non-papillary) stricture crossing.

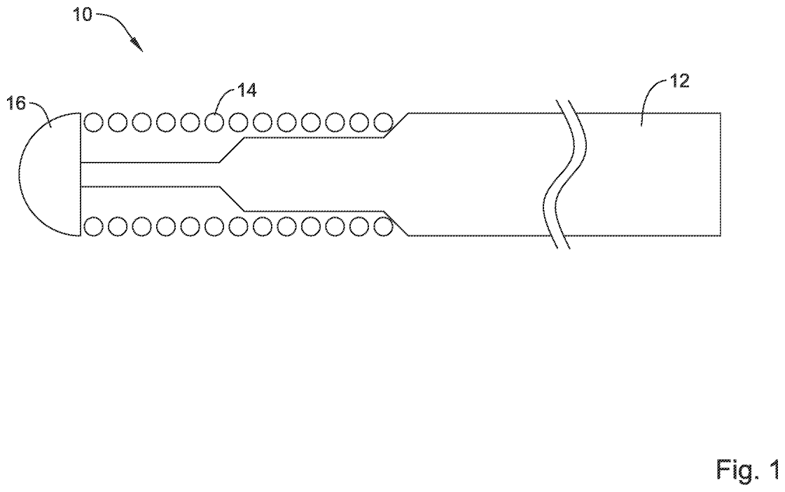

FIG. 1 schematically depicts an example medical device 10, which may take the form of a guidewire. The guidewire 10 can have a number of different structural configurations. For example, the guidewire 10 may include a core wire 12 and tip region including a coil 14 and a distal tip 16. In other instances, the guidewire 10 may include a polymer tip (not shown). In general, the guidewire 10 may be configured to be advanced toward a stricture along the pancreatic and/or biliary tract. This may include using an antegrade approach to navigate the guidewire 10 to a position along the pancreatic and/or biliary tract.

In at least some instances, the guidewire 10 may be relatively flexible, for example so the guidewire 10 can be advanced through body lumens/tissue in an atraumatic manner. The flexible nature of the guidewire 10 may make it challenging to cross a stricture such as a stricture along the pancreatic and/or biliary tract. It may be desirable to utilize a support structure, for example a structure that can provide additional stiffness and/or pushability, in order to advance the guidewire 10 past a stricture such as a stricture along the pancreatic and/or biliary tract.

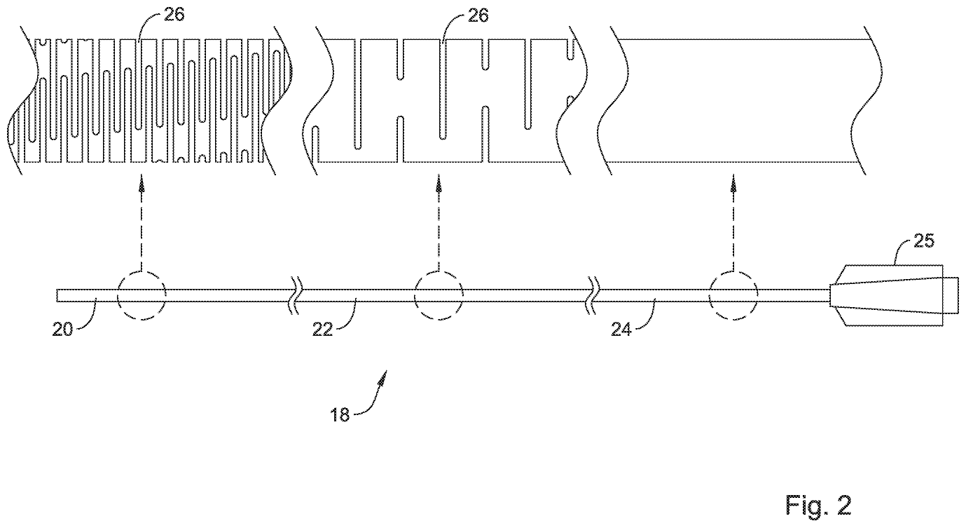

FIG. 2 is a side view of an example tubular support sheath 18 that can be used in conjunction with the guidewire 10, for example to help advance the guidewire 10 past a stricture along the pancreatic and/or biliary tract. The tubular support sheath 18 may generally be slidable along the guidewire 10 and the tubular support sheath 18 may include a distal end region 20, a body region 22, and a proximal end region 24. A hub 25 may be coupled to the proximal end region 24.

The tubular support sheath 18 may have a plurality of slots 26 formed therein. In some instances, the slots 26 may vary in density along the length of the tubular support sheath 18. For example, the distal end region 20 may include a relatively dense slot distribution with a relatively high number of slots 26 per unit length. The body region 22 may have a lower slot density (e.g., fewer slots 26 per unit length than the distal end region 20). The proximal end region 24 may have an even lower slot density than the body region 22. In some instances, the proximal end region 24 may be free of slots 26. The slot density may transition along the length of the tubular support sheath 18, for example from the body region 22 to the distal end region 20. This may include a gradual transition, a stepped transition, and/or the like.

Various arrangements and configurations of slots 26 are contemplated. In some embodiments, at least some, if not all of the slots 26 are disposed at the same or a similar angle with respect to the longitudinal axis of the tubular support sheath 18. As shown, the slots 26 can be disposed at an angle that is perpendicular, or substantially perpendicular, and/or can be characterized as being disposed in a plane that is normal to the longitudinal axis of the tubular support sheath 18. However, in other embodiments, the slots 26 can be disposed at an angle that is not perpendicular, and/or can be characterized as being disposed in a plane that is not normal to the longitudinal axis of the tubular support sheath 18. Additionally, a group of one or more slots 26 may be disposed at different angles relative to another group of one or more slots 26. The distribution and/or configuration of the slots 26 can also include, to the extent applicable, any of those disclosed in U.S. Pat. Publication No. US 2004/0181174, the entire disclosure of which is herein incorporated by reference.

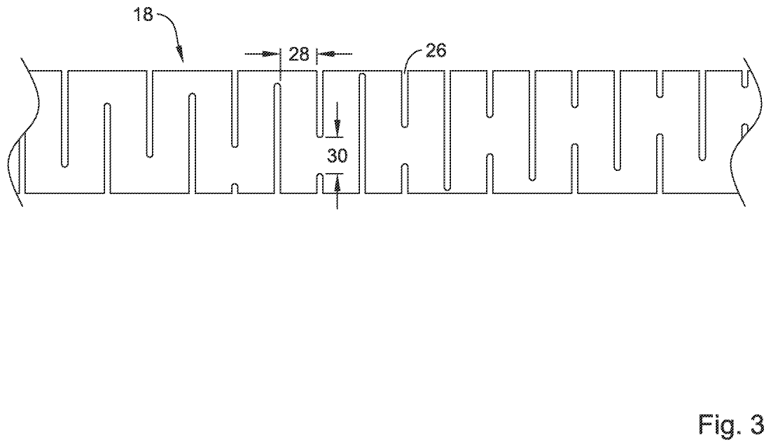

The slots 26 may be provided to enhance the flexibility of the tubular support sheath 18 while still allowing for suitable torque transmission characteristics. As shown in FIG. 3, the slots 26 may be formed such that one or more rings 28 and one or more beams 30 are formed in the tubular support sheath 18, and such rings 28 and beams 30 may include portions of the tubular support sheath 18 that remain after the slots 26 are formed in the tubular support sheath 18. Such an interconnected structure may act to maintain a relatively high degree of torsional stiffness, while maintaining a desired level of lateral flexibility. In some instances, at least some of the slots 26 are arranged so that the height or length (e.g. the dimension in the direction of the longitudinal axis of the tubular support sheath 18) of the rings 28 is substantially equal to the height or width (e.g. the dimension in the circumferential direction about the tubular support sheath 18) of the beams 30 is substantially equal.

In some embodiments, some adjacent slots 26 can be formed such that they include portions that overlap with each other about the circumference of the tubular support sheath 18. In other embodiments, some adjacent slots 26 can be disposed such that they do not necessarily overlap with each other, but are disposed in a pattern that provides the desired degree of lateral flexibility.

Additionally, the slots 26 can be arranged along the length of, or about the circumference of, the tubular support sheath 18 to achieve desired properties. For example, adjacent slots 26, or groups of slots 26, can be arranged in a symmetrical pattern, such as being disposed essentially equally on opposite sides about the circumference of the tubular support sheath 18, or can be rotated by an angle relative to each other about the axis of the tubular support sheath 18. Additionally, adjacent slots 26, or groups of slots 26, may be equally spaced along the length of the tubular support sheath 18, or can be arranged in an increasing or decreasing density pattern, or can be arranged in a non-symmetric or irregular pattern. Other characteristics, such as slot size, slot shape, and/or slot angle with respect to the longitudinal axis of the tubular support sheath 18, can also be varied along the length of the tubular support sheath 18 in order to vary the flexibility or other properties. In other embodiments, moreover, it is contemplated that the portions of the tubular support sheath 18, such as a proximal section, or a distal section, or the entire the tubular support sheath 18, may not include any such slots 26.

The slots 26 may be formed in groups of two, three, four, five, or more slots 26, which may be located at substantially the same location along the axis of the tubular support sheath 18. Alternatively, a single slot 26 may be disposed at some or all of these locations. Within the groups of the slots 26, there may be included slots 26 that are equal in size (e.g., span the same circumferential distance around the tubular support sheath 18). In some of these as well as other embodiments, at least some slots 26 in a group are unequal in size (e.g., span a different circumferential distance around the tubular support sheath 18). Longitudinally adjacent groups of slots 26 may have the same or different configurations. For example, some embodiments of the tubular support sheath 18 include slots 26 that are equal in size in a first group and then unequally sized in an adjacent group. It can be appreciated that in groups that have two slots 26 that are equal in size and are symmetrically disposed around the tube circumference, the centroid of the pair of beams 30 is coincident with the central axis of the tubular support sheath 18. Conversely, in groups that have two slots 26 that are unequal in size and whose centroids are directly opposed on the tube circumference, the centroid of the pair of beams 30 can be offset from the central axis of the tubular support sheath 18. Some embodiments of the tubular support sheath 18 include only slot groups with centroids that are coincident with the central axis of the tubular support sheath 18, only slot groups with centroids that are offset from the central axis of the tubular support sheath 18, or slot groups with centroids that are coincident with the central axis of the tubular support sheath 18 in a first group and offset from the central axis of the tubular support sheath 18 in another group. The amount of offset may vary depending on the depth (or length) of the slots 26 and can include other suitable distances.

The slots 26 can be formed by methods such as micro-machining, saw-cutting (e.g., using a diamond grit embedded semiconductor dicing blade), electron discharge machining, grinding, milling, casting, molding, chemically etching or treating, or other known methods, and the like. In some such embodiments, the structure of the tubular support sheath 18 is formed by cutting and/or removing portions of the tube to form the slots 26. Some example embodiments of appropriate micromachining methods and other cutting methods, and structures for tubular members including slots and medical devices including tubular members are disclosed in U.S. Pat. Publication Nos. 2003/0069522 and 2004/0181174-A2; and U.S. Pat. Nos. 6,766,720; and 6,579,246, the entire disclosures of which are herein incorporated by reference. Some example embodiments of etching processes are described in U.S. Pat. No. 5,106,455, the entire disclosure of which is herein incorporated by reference. It should be noted that the methods for manufacturing guidewire 110 may include forming the slots 26 the tubular support sheath 18 using these or other manufacturing steps.

In at least some embodiments, the slots 26 may be formed in tubular member using a laser cutting process. The laser cutting process may include a suitable laser and/or laser cutting apparatus. For example, the laser cutting process may utilize a fiber laser. Utilizing processes like laser cutting may be desirable for a number of reasons. For example, laser cutting processes may allow the tubular support sheath 18 to be cut into a number of different cutting patterns in a precisely controlled manner. This may include variations in the slot width, ring width, beam height and/or width, etc. Furthermore, changes to the cutting pattern can be made without the need to replace the cutting instrument (e.g., blade).

The tubular support sheath 18 may include a number of additional structural features/variations. For example, the tubular support sheath 18 may define a central lumen extending therethrough. The lumen may be used, for example, for passage of the guidewire 10, infusion of materials such as contrast media, combinations thereof, and/or the like. In some instances, an inner member or liner may be disposed along the inner surface of the tubular support sheath 18 that defines the lumen. In other instances, the inner surface of the tubular support sheath 18 may be free of an inner member or liner. In some instances, an outer layer or outer tubular member may be disposed along the outer surface of the tubular support sheath 18. The outer layer may be formed from a polymer and/or an insulating material, which may allow the tubular support sheath 18 to be used during electrical/RF interventions. In some instances, a tip member may be coupled to the distal end region 20 of the tubular support sheath 18. The tip member may include a coil (e.g., formed from a round wire, a ribbon or flat wire, and/or the like) and, in some instances, a sleeve disposed along or encapsulating the coil. In some instances, a relatively short tubular tip member may be coupled to the distal end region 20. The tubular tip member may include a radiopaque marker. These are just examples. Other structural features are contemplated.

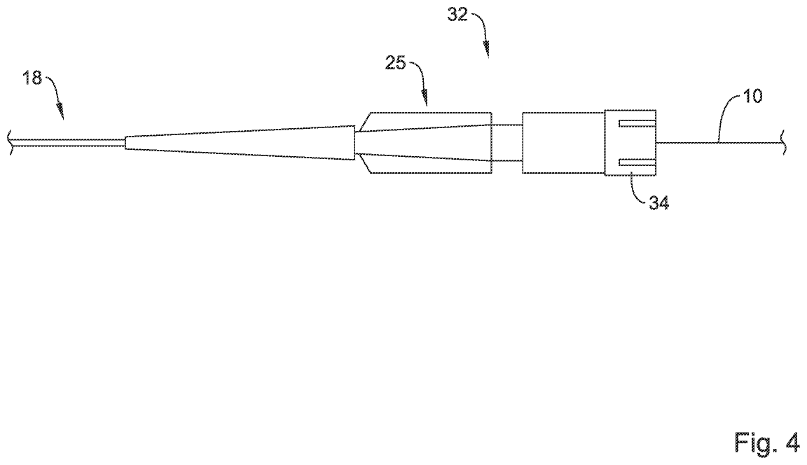

When used together, the tubular support sheath 18 can provide structural support to the guidewire 10, for example when taking an antegrade approach to navigating toward and beyond a stricture. FIG. 4 illustrates the guidewire 10 and the tubular support sheath 18 together as a medical device system 32 to antegrade stricture crossing along the pancreatic and/or biliary tract. In addition, while approaching and/or crossing a stricture, it may be desirable to secure the axial position of the guidewire 10 relative to the tubular support sheath 18. In some instances, the hub 25 may include a locking member 34 for securing the axial position of the guidewire 10 relative to the tubular support sheath 18. The form of the locking member 34 may vary. In some instances, the locking member 34 may include a collet, a Tuohy Borst connector that seals around the guidewire 10, and/or the like.

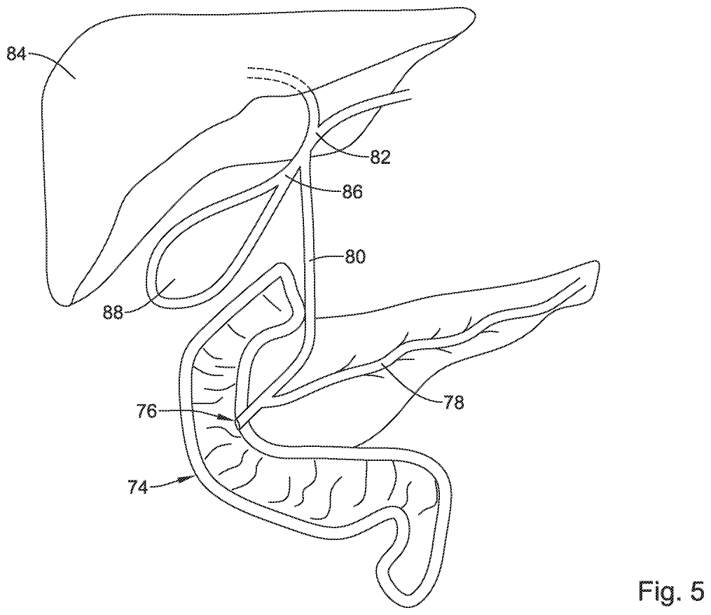

FIG. 5 illustrates an overview of the biliary system or tree. A portion of the duodenum 74 is shown. The papilla of Vater 76 (e.g., also known as the ampulla of Vater or simply the papilla) is located at the illustrated portion of the duodenum 74. The papilla 76 generally forms the opening where the pancreatic duct 78 and the common bile duct 80 can empty into the duodenum 74. The hepatic ducts, denoted by the reference numeral 82, are connected to the liver 84 and empty into the bile duct 80. Similarly, the cystic duct 86, being connected to the gall bladder 88, also empties into the bile duct 80. In general, an endoscopic or biliary procedure may include advancing a medical device to a suitable location along the biliary tree and then performing the appropriate intervention.

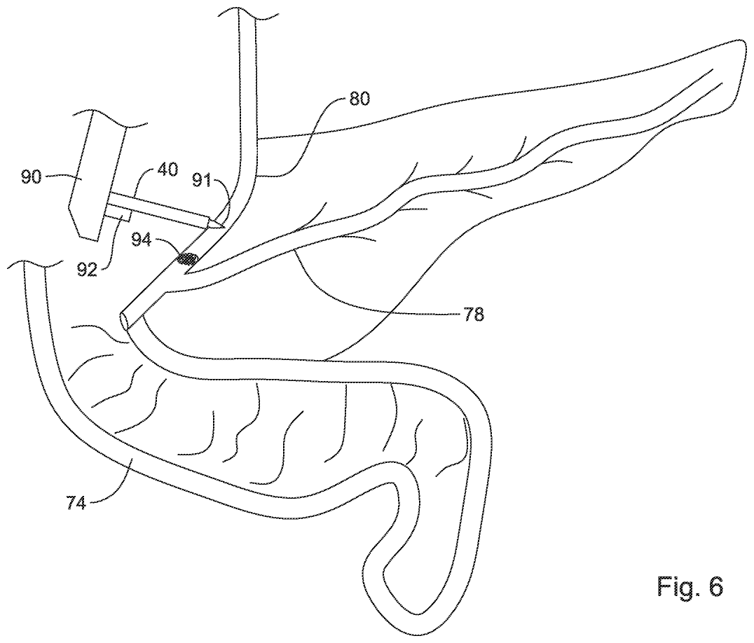

In some instances, it may be desirable to navigate the guidewire 10 and/or the tubular support sheath 18 past a stricture 94 along the pancreatic and/or biliary tract. For example, FIG. 6 depicts an endoscope 90 extending into the duodenum 74. In some instances, a catheter or introducer sheath 40 may be advanced through the endoscope 90 (e.g., through a channel formed in the endoscope 90). The introducer sheath 40 may be directed toward the wall of the duodenum 74 with the elevator 92 of the endoscope 90. In some instances, a needle/sharp 91 may be disposed within the introducer sheath 40. The needle/sharp 91 may help to pierce through the wall of the duodenum 74, through tissue, and into a position along the pancreatic and/or biliary tract adjacent to a stricture 94. In this example, the stricture 94 is disposed along the bile duct 80.

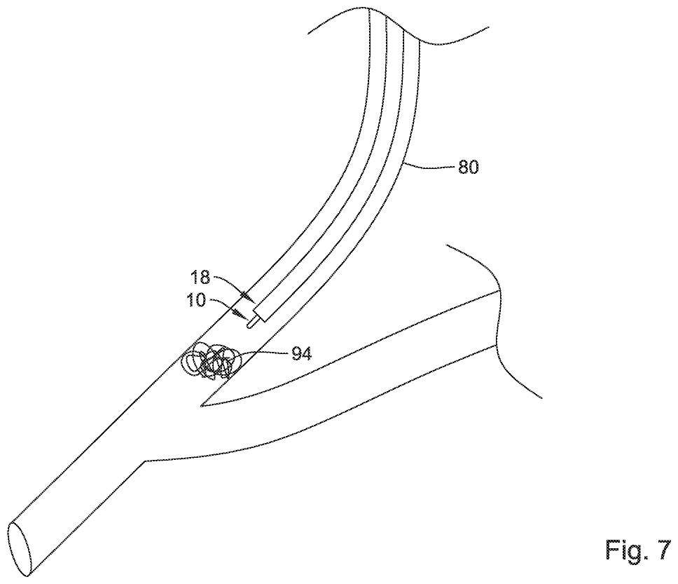

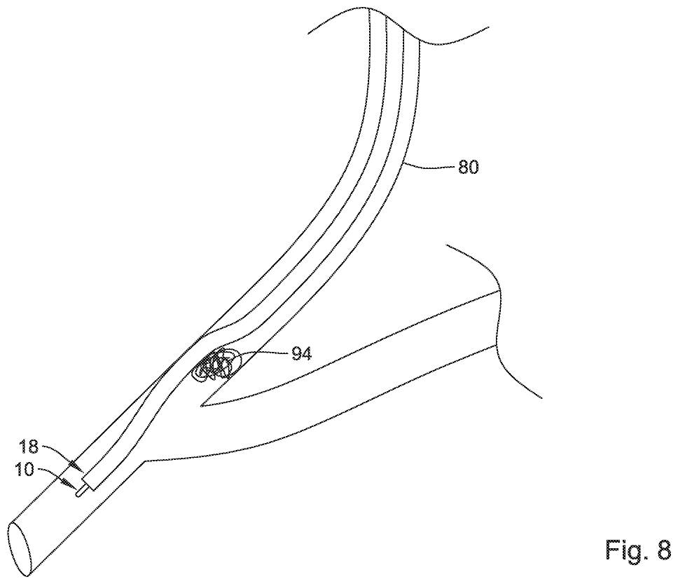

In order to cross the stricture 94, the guidewire 10, while being structurally supported by the tubular support sheath 18, may be navigated toward the stricture 94 as depicted in FIG. 7. The structural support of the tubular support sheath 18 may aid advancing the guidewire 10 beyond the stricture 94 as depicted in FIG. 8. In at least some instances, the hub 25 (e.g., the locking member 34) may be used to secure the axial position of the guidewire 10 relative to the tubular support sheath 18 while advancing/crossing the guidewire 10 beyond the stricture 94.

The materials that can be used for the various components of the devices disclosed herein may include those commonly associated with medical devices. For simplicity purposes, the following discussion makes reference to the tubular support sheath 18. However, this is not intended to limit the devices and methods described herein, as the discussion may be applied to other similar tubular members and/or components of tubular members or devices disclosed herein.

The tubular support sheath 18 may be made from or otherwise includes a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A system for treating a stricture, the system comprising:
a guidewire for antegrade stricture crossing along the biliary and/or pancreatic tract, the guidewire having a distal end region and a proximal end region;
a hub coupled to the proximal end region;
a tubular sheath slidably disposed along the guidewire, the tubular sheath having a plurality of slots formed therein; and
wherein the hub is configured to secure the axial position of the tubular sheath relative to the guidewire.

2. The system of claim 1, further comprising an introducer sheath, wherein the guidewire is configured to be advanced through the introducer sheath.

3. The system of claim 1, further comprising an endoscope, wherein the guidewire is configured to be advanced through the endoscope.

4. The system of claim 1, wherein the tubular sheath has a proximal portion having a first slot density and a distal portion having a second slot density greater than the first slot density.

5. The system of claim 1, wherein the tubular sheath has a proximal region free of slots.

6. The system of claim 1, wherein the plurality of slots define a plurality of beams including a first beam and a plurality of rings including a first ring in the tubular sheath, wherein the first beam has a first beam height, wherein the first ring has a first ring length, and where the first beam height is substantially equal to the first ring length.

7. The system of claim 1, wherein the hub includes a locking member for securing the axial position of the tubular sheath relative to the guidewire.

8. The system of claim 1, wherein the hub is secured to the tubular sheath.

9. The system of claim 1, wherein the tubular sheath defines a contrast lumen for infusing contrast media.

10. A method for treating a stricture along the biliary and/or pancreatic tract, the method comprising:
advancing an introducer sheath through a working channel of an endoscope to a position along the biliary and/or pancreatic tract to a position adjacent to a stricture;
advancing a multi-part guidewire system through the introducer sheath, the multi-part guidewire system comprising:
a guidewire having a distal end region and a proximal end region,
a hub coupled to the proximal end region,
a tubular sheath slidably disposed along the guidewire, the tubular sheath having a plurality of slots formed therein, and
wherein the hub is configured to secure the axial position of the tubular sheath relative to the guidewire;
shifting the axial position of the tubular sheath relative to the guidewire; and
advancing the multi-part guidewire system past the stricture.

11. The method of claim 10, wherein advancing an introducer sheath through a working channel of an endoscope to a position along the biliary and/or pancreatic tract to a position adjacent to a stricture includes piercing through tissue.

12. The method of claim 10, wherein advancing an introducer sheath through a working channel of an endoscope to a position along the biliary and/or pancreatic tract to a position adjacent to a stricture includes an antegrade approach toward the stricture.

13. The method of claim 10, further comprising removing the tubular sheath from the guidewire.

14. The method of claim 10, further comprising advancing a treatment device over the guidewire.

15. The method of claim 10, wherein a portion of the guidewire extends distally beyond a distal end of the tubular sheath, and wherein shifting the axial position of the tubular sheath relative to the guidewire includes lengthening the portion of the guidewire that extends distally beyond the distal end of the tubular sheath.

16. The method of claim 10, wherein a portion of the guidewire extends distally beyond a distal end of the tubular sheath, and wherein shifting the axial position of the tubular sheath relative to the guidewire includes shortening the portion of the guidewire that extends distally beyond the distal end of the tubular sheath.

17. A system for treating a stricture along the biliary and/or pancreatic tract, the system comprising:
a support sheath having a distal end region, a proximal end region, and having a plurality of slots formed therein;
a hub secured to the proximal end region;
a guidewire slidably disposed within the support sheath; and
wherein the hub is configured to secure the axial position of the guidewire relative to the support sheath.

18. The system of claim 17, wherein the support sheath has a proximal portion having a first slot density and a distal portion having a second slot density greater than the first slot density.

19. The system of claim 17, wherein the support sheath has a proximal region free of slots.

20. The system of claim 17, wherein the plurality of slots define a plurality of beams including a first beam and a plurality of rings including a first ring in the tubular sheath, wherein the first beam has a first beam length, wherein the first ring has a first ring length, and where the first beam length is substantially equal to the first ring length.
